(19) United States
(12) Patent Application Publication
(10) Pub. No.: US 2006/0257368 A1
(43) Pub. Date: Nov. 16, 2006

(54) Devices and methods for improved viability of cells

(75) Inventor: Xuejun Wen, Mount Pleasant, SC (US)

Correspondence Address: DORITY & MANNING, P.A., POST OFFICE BOX 1449, GREENVILLE, SC, 29602-1449, US

(73) Assignee: Clemson University

(21) Appl. No.: 11/416,635

(22) Filed: May 3, 2006

Related U.S. Application Data
(60) Provisional application No. 60/677,179, filed on May 3, 2005.

(51) Int. Cl. A61K 48/00; A61K 31/7034; C12M 3/00; A61M 31/00

(52) U.S. Cl. 424/93.2; 514/25; 435/289.1; 604/500

(57) ABSTRACT

Disclosed are devices and methods for improved viability of cells. In one embodiment of the present disclosure, a method for improved viability of cells is provided. The method includes providing a device having a body, an access port, and an insert. The body includes a selectively permeable hollow fiber membrane and the insert includes one or more scar suppression agents. The device is implanted whereby the scar suppression agent is delivered.

### CROSS REFERENCE TO RELATED APPLICATIONS

The present application claims benefit of U.S. Provisional Application Ser. No. 60/677,179 having a filing date of May 3, 2005 and U.S Provisional Application Ser. No. ??/?????? having a filing date of Apr. 26, 2006.

### BACKGROUND

Cell regeneration, implantation, and transplantation are very promising strategies for the treatment of many diseases and injuries including Parkinson's disease, Huntington's disease, Alzheimer's disease, multiple sclerosis, diabetes, stroke, central nervous system injury and the like.

Human stem cells have become an important cell source because of their remarkable self-renewal and differentiation properties. Recent progress in human stem cell research indicates the enormous potential in transplanting human stem cells or their derived cells for the treatment of many diseases and injuries. Cell regeneration, particularly following central nervous system injury, is also a desireable treatment option. However, the success of such approaches is greatly hampered by poor cell survival.

Current approaches fail to significantly improve viabilty of cells because they do not adequately address problems at the regeneration, implantation, and/or transplantation sites. Issues at the local tissue sites include low oxygen level, low nutrient supply, high level release of cytokines and chemokines during acute and sub-acute inflammatory responses, scar formation, as well as immune rejection from the host. Thus, a need exists for approaches that resolve such issues and thereby improves the viability of cells.

### SUMMARY

The present disclosure recognizes and addresses the foregoing needs as well as others. In one embodiment of the present disclosure, a method for improved viability of cells is provided. The method includes providing a device having a body, an access port, and an insert. The body includes a selectively permeable hollow fiber membrane and the insert includes one or more scar suppression agents. The device is implanted whereby the scar suppression agent is delivered.

In certain embodiments, the scar suppression agent may include 4-nitrophenyl-β-D-xylopyranoside. In some embodiments, the selectively permeable hollow fiber membrane may be degradable. In certain embodiments, the insert includes one or more transplanted cells. In some embodiments, the transplanted cells may include stem cells. In some embodiments, the method may also include developing immune tolerance in the transplanted cells.

In another embodiment of the present disclosure, a method for improved viability of cells is provided. The method includes providing a device having a body, an access port, and an insert. The body includes a selectively permeable hollow fiber membrane and the insert includes one or more scar suppression agents and one or more transplanted cells. The device is implanted whereby the scar suppression agent and the transplanted cells are delivered.

In still another embodiment of the present disclosure, a device for delivery of scar suppression agents is provided. The device includes a body having a proximal end and a distal end and defining a cavity. An access port is located at the proximal end of the body and an insert is configured to be inserted into the cavity of the body. The insert includes one or more scar suppression agents.

In yet another embodiment of the present disclosure, a device for delivery of scar suppression agents is provided. The device includes a body having a proximal end and a distal end and defining a cavity. An access port is located at the proximal end of the body and an insert is configured to be inserted into the cavity of the body. The insert includes one or more scar suppression agents and one or more transplanted cells.

### DESCRIPTION OF THE DRAWINGS

A full and enabling disclosure, including the best mode thereof to one of ordinary skill in the art, is set forth more particularly in the remainder of the specification, including reference to the accompanying figures in which:

FIG. 1 illustrates a delivery device in accordance with one embodiment of the present disclosure;

FIG. 2 illustrates survival rates of transplanted cells by location in accordance with one embodiment of the present disclosure;

FIG. 3 illustrates survival rates of transplanted neuron cells by location in accordance with one embodiment of the present disclosure;

FIG. 4 illustrates an injury as may be treated in accordance with one embodiment of the present disclosure;

FIG. 5 illustrates a tubular scaffold in accordance with one embodiment of the present invention;

FIG. 6 illustrates a blank control hollow fiber membrane compared to a constant release of PNPX in accordance with one embodiment of the present disclosure;

FIG. 7 illustrates toxicity of PNPX to human mesenchymal stem cells;

FIG. 8 illustrates accumulated PNPX release from a PNPX-loaded HFM in accordance with one embodiment of the present disclosure; and

FIG. 9 illustrates 24 hour release from PNPX-loaded HFMs in accordance with one embodiment of the present disclosure.

### DETAILED DESCRIPTION

It is to be understood by one of ordinary skill in the art that the present discussion is a description of exemplary embodiments only, and is not intended as limiting the broader aspects of the present disclosure, which broader aspects are embodied in the exemplary construction.

In general, the present disclosure is directed to devices and methods for improved viability of cells. In particular, several approaches are described which greatly enhance the long-term viability and functionality of cells in vivo. Although the approaches described herein are directed to in vivo applications, ex vivo applications are equally applicable to the devices and methods of the present disclosure. More particularly, the devices and methods include techniques that may each be used separately or in combination.

Typically, the low survival rate of cells or tissue may be caused by a number of factors. Some of these factors may include:

1) low oxygen level and low nutrient supply due to vascular network damage and edema to local tissue as a result of cell implantation/transplantation procedures;

2) high level release of cytokines and/or chemokines during acute and sub-acute inflammatory responses which may result from cell implantation/transplantation procedures;

3) immune response from the host; and

4) scar formation after acute inflammatory response.

With regard to conventional implantation and transplantation procedures, penetrating injury may be introduced to tissue immediately before such procedures, resulting in instant recruitment of multiple inflammatory cell types at the implantation/transplantation site. Such inflammatory cell types may jeopardize the survival of implanted/transplanted cells. Implantation/transplantation into host tissue can induce injury that elicits a wound healing response. Without appropriate intervention, injury eventually leads to the formation of an extracellular matrix (ECM)-rich scar tissue that is believed to be associated with impeding oxygen and nutrient transport between the implanted/transplanted cells and the host tissue. In addition, ECM molecules can inhibit cell regeneration.

Low oxygen level and low nutrient supply due to vascular network damage and edema to local tissue, host tissue inflammatory response, scar formation, and immune response are factors when considering regenerated, implanted, and/or transplanted cell survival and functionality. To overcome such barriers, techniques have been developed to ultimately enhance the long-term viability and functionality of cells in vivo.

In this regard, a non-limiting exemplary list of cell types may include any types of cells that may be either regenerated, implanted, or transplanted into human body or utilized in vivo, such as undifferentiated stem cells, precursor cells, neurons (and projections therefrom, such as axons), oligodendrocytes, Schwann cells, olfactory ensheathing cells, bone marrow cells, endothelial cells, osteoblasts, cardiomyocytes, skeletal myocytes, beta cells, epithelial cells, endothelial cells, and the like.

In one embodiment, a scar-free space can be created in which to locate the cells. As used herein, a scar or scarring can refer to the dense connective tissue forming a scar. In this regard, scar-free can refer to reduced development of scar tissue. A scar-free space allows for improved long-term survival and functionality of relocated cells placed in the space through subsidence of acute and sub-acute inflammatory responses prior to the implantation/transplantation of functional cells among other possible factors. For instance, a scar-free space can be created through implantation or location of a biodegradable spacer encapsulated with scar suppressing or scar dissolving agents at or near the implantation/transplantation site prior to the implantation or transplantation of the functional cells.

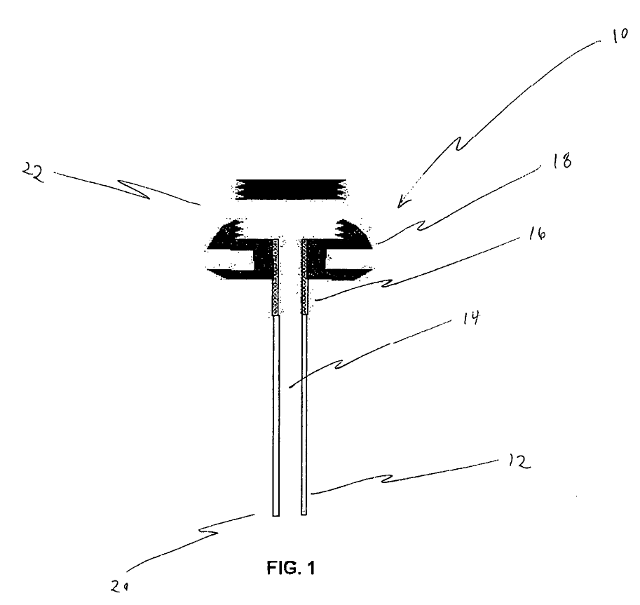

Referring to FIG. 1, a structure 10 (also referred to herein as a spacer) in accordance with one embodiment of the present disclosure is shown. As described herein, the spacer is biodegradable. However, it should be understood that the spacer can also not be degradable and can be reused.

One embodiment of a biodegradable spacer 10 can include a body 12 formed from a permeable tubular structure. The body 12 can be formed, for example, from a selectively permeable hollow fiber membrane. Selectively-permeable hollow fiber membranes can be fabricated as would be known in the art. For example, phase inversion method may be utilized to fabricate a selectively permeable hollow fiber membrane.

The biodegradable spacer 10 can have a generally linear, cylindrical shape with a distal end 20 and proximal end 22. However, the body 12 is not limited to a generally linear, cylindrical shape and other shapes may be utilized as well. The body 12 can have an internal volume suitable to hold an amount of scar suppressing or scar dissolving agent(s) within the lumen 14 and which can also provide a permeable boundary with sufficient surface area through which the agent can be delivered. In some embodiments, the body 12 may have variable permeability areas including areas of non-permeability. In some embodiments, scar suppressive agents are encapsulated inside a tubular structure while in other embodiments, scar suppressive agents are fabricated into the degradable material forming the body 12 for delivery.

Suitable scar suppressing agents can include biologically active chemical agents, such as methylpredisone or cyclosporin-A (CsA), ethodium bromide, scar suppressive peptides/ proteins, 4-nitrophenyl-β-D-xylopyranoside, and/or special cells, such as scar suppressive immature astrocytes or their derived factors, and immunosuppressive Sertoli cells. However, any other scar suppressing agents as known in the art would also be suitable. Scar dissolving agents may include chondroitinase ABC, collagenase, trypsin, elastase, or other agents as would be known in the art. In some embodiments, immature astrocytes may be utilized as a scar suppressive agent. Such scar suppressing agents may be utilized either alone or in combination.

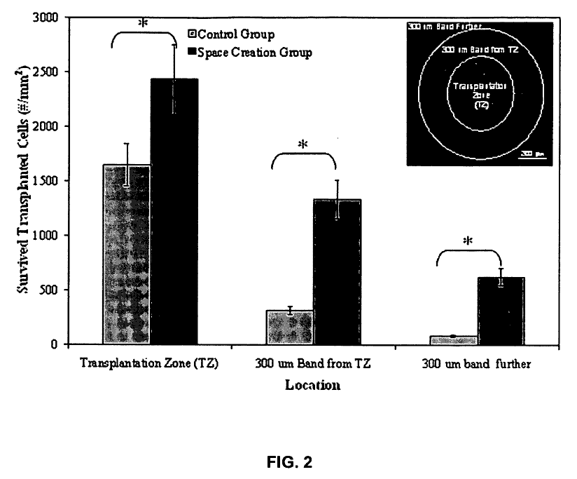

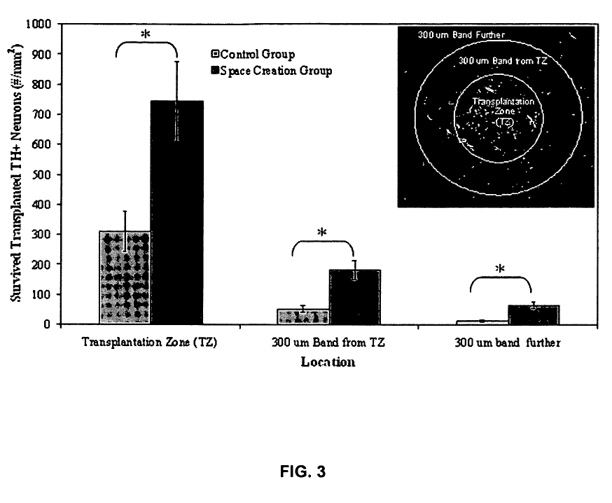

Referring to FIGS. 2-3, graphs illustrating survival rates of transplanted cells and transplanted neuron cells are depicted. A scar free interface improves the oxygen and nutrient supply to implanted/transplanted cells and can promote migration of implanted/transplanted cells, thereby improving the integration of the relocated cells with the host tissue or surrounding environment (in an ex vivo application). A scar free interface can be beneficial with transplantation therapy for the treatment of neurodegenerative diseases, such as Parkinson's disease, Huntington's disease, Alzheimer's disease, and multiple sclerosis. However, it should be understood that the scar free interface as described herein may be beneficial in the treatment of a wide array of other disorders.

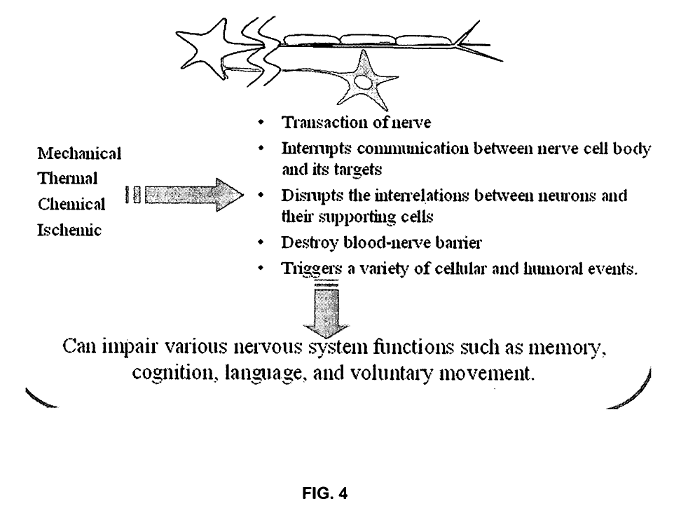

For example, following adult central nervous system (CNS) injury (see FIG. 4), resident cells and blood-derived inflammatory cells can become activated and migrate to a lesion site, eventually forming a dense glial scar that is believed to play a significant role in inhibiting CNS regeneration. Irrespective of the type and extent of the injury, the great difficulty in CNS regeneration following injury is largely due to the disruption of organized architecture at the lesion site. An organized architecture is important in guiding cell migration, inducing appropriately aligned morphology and directing axonal outgrowth. In a typical nervous system with the existence of normal organized architecture, neurites are able to elongate and grow long distances. However, when such architecture is destroyed, neurite outgrowth is terminated.

The inhibitory nature of the glial scar is due not only to the existence of inflammatory cell types, but also to the ECM molecules that are derived from them. In particular, chrondroitin sulfate proteoglycan (CSPG), a family of ECM molecules secreted by astrocytes can upregulate in the glial scar and can inhibit neurite extension. Other factors contributing to the inhibitory environment following injury include oligodendrocytes and myelin-associated glycoprotein, reactive microglial cells and macrophages, meningeal fibroblasts, cysts, a lack of growth promoting factors, and the like. The digestion of CSPG can enhance axonal regeneration after spinal cord injury. Therefore, blocking CSPG biosynthesis can ameliorate CNS glial scar formation and to promote neural regeneration.

In some embodiments, one or more scar suppressing agents can be used to suppress glial scar formation in the central nervous system. Suppressing glial scar formation can improve neural regeneration after spinal cord and brain injury. In some embodiments, one or more scar suppressing agents can also be used to combine with cell implantation/transplantation strategies, such as stem cell transplantation, to improve the survival, migration, and integration with host tissue.

In some embodiments, a tubular sleeve structure is placed across an injury site to create a scar-free controlled regeneration environment. The tubular sleeve can prevent penetration of the scar tissue into the lumen where regenerating axons grow. Cells or growth factors can be entubulated into the sleeve to facilitate the axonal outgrowth.

In some embodiments, the tubular sleeve is formed from a selectively-permeable hollow fiber membrane. In such embodiments, selectively-permeable hollow fiber membranes can be utilized to promote such axonal regeneration both in vitro and in vivo. Such selectively-permeable hollow fiber membranes can reduce the infiltration of fibrous tissue, provide a conduit for the diffusion of neurotropic and neurotrophic factors, increase the concentration of endogenous proteins inside the channel, and present a barrier to selectively permit or inhibit the diffusion of macromolecules between the tubular structure and the surroundings.

As described previously, in some embodiments of the present disclosure, the chemical, 4-nitrophenyl-β-D-xylopyranoside (PNPX), can be utilized as a scar suppression agent.

In some embodiments, PNPX can suppress the glial scarring response in injured CNS tissue. In some embodiments, PNPX can be used for the treatment of spinal cord injury, brain trauma, and also be combined with cell implantation/transplantation to improve the survival, migration, and functional integration of transplanted cells with host tissue.

Various suitable methods may be utilized to implant a spacer encapsulated with scar-suppressing or dissolving agents. In some embodiments, a spacer encapsulated with scar-suppressing or dissolving agents can be implanted into the appropriate location in the body tissue to ameliorate tissue inflammatory response and suppress scar formation. In some embodiments, the spacer can be implanted in brain tissue to ameliorate brain tissue inflammatory response and suppress scar formation. A spacer can be implanted into almost any location in the brain, including the cortex, striatum, substantia nigra, and corpus collosum. In addition to the brain, the spacer can be implanted into middle or inner ear, etc. The spacer may be formed to any size and shape and implanted in any location where cell transplant may be desirable. Also, the spacer can be biodegradable.

In some embodiments, the spacer can be connected to a re-accessible port. For example, referring again to FIG. 1, in one embodiment, the biodegradable spacer can be connected via a short segment of non-degradable, non-permeable tube 16 connected to a re-accessible port 18, which can be anchored to the surface of a body or 3-D matrix to allow repeatable access to the scar-free space created by biodegradable spacer 10 inside the tissue. For example, in one embodiment, the re-accessible port 18 can be anchored on the skull to allow repeatable access to the scar-free space created by the biodegradable spacer inside the brain tissue. In one embodiment, the re-accessible port 18 can be formed from a biocompatible flexible polymer, such as elastomer. In this regard, biocompatible refers to a material such as that forming an access port that is substantially non-immunogenic and does not lead to the formation of toxic, potentially immunogenic reaction products at the tissue site of administration. The overall size and shape of the re-accessible port 18 is not particularly limited but can generally be sufficient to anchor the spacer to the general area where cell transplantation is desired.

In another embodiment, the distal end 22 of the body 12 may be removeably sealed such as with biodegradable glue or the like. The seal may be shaped with any geometry, for example pointed, or simply blunt. It can serve to seal the body 12 of the spacer 10 and prevent scar-suppressing or scar-dissolving agent from exiting the device from the distal end 22 and thus encourage exit of agent through the body 20 to achieve controlled and slow release therapeutic agents.

After termination of acute and sub-acute inflammatory responses (often within 2-4 weeks), in some embodiments, the biodegradable spacer can dissolve and a scar-free space can be formed. This can improve the growth and development of functional cells, such as stem cells or their derived cells, through the re-accessible port into the scar-free space without causing injury to body tissue.

Again, although the spacer is described in accordance with certain aspects of the present disclosure as being biodegradable, it should be understood that in certain embodiments, the spacer does not dissolve and can be used repeatedly. In some embodiments, additional scar-suppressing agent and/or cells can be added to the body 12 via the re-accessible port 18.

Yet another embodiment involves increasing hypoxia tolerance prior to the cells being transplanted. In this embodiment, cells may be exposed to hypoxia simulating agents before the transplantation procedure so as to increase their ability of glucose uptake and glycolytic metabolism. These agents can be supplied to culture media of the cells for several minutes up to several days before the transplantation into the body or new environment. Once such agents are supplied the culture media, cells will absorb or otherwise process the drug and mimic a low oxygen condition. Increased glucose uptake and glycolytic metabolism is an important compensatory mechanism that allows ATP production to be maintained during hypoxia. Such an approach can also increase resistance of the transplanted cells to certain metabolic poisons. For instance, in one embodiment, hypoxia-simulating agents can be added to cultured cells several hours to several days prior to transplantation. Suitable hypoxia-simulating agents may include for example, dimethyloxalylglycine and ethyl 3,4-dihydroxybenzoate. However, any other suitable hypoxia-simulating agents as would be known to one in the art may also be utilized. The resulting transplanted cells can be adjusted to better withstand a deficiency of oxygen and, thus, more resistant to transplantation or relocation procedures.

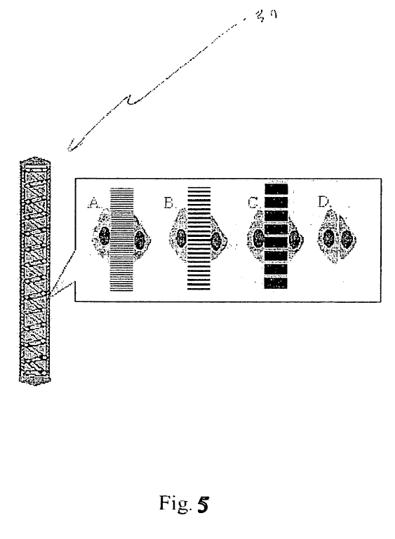

Still another embodiment involves developing immune tolerance in the transplanted cells. Referring to FIG. 5, in one embodiment, immune tolerance may be developed by increasing exposure of the transplanted cells to the host tissue over time by using a biodegradable selectively-permeable HFM system 30. System 30 can allow host tissue to develop an immune ignorance to the transplanted cells. In some embodiments, cells can be encapsulated in the tubular structure (as described above) and implanted into the host tissue, such as subcutaneous, muscle, peritoneal, and the like. In some embodiments, with the degradation of the tubular structure (as illustrated sequentially in A-D of FIG. 5), transplanted cells can be gradually exposed to the host immune system. In some embodiments, the host immune system may ignore the antigens presented by the transplanted cells and may not reject the transplanted cells over the duration of the degradation.

In some embodiments, prior to functional cell transplantation, a small number of cells, e.g., 1,000 to 10,000 cells, can be encapsulated into a selective permeable biodegradable HFM device with the biodegradable HFM having a molecular weight cut off ranging from 20K to 70K. The biodegradable HFM can then be implanted in the body tissue. Pores on the HFM can grow larger during the degradation process of HFM device resulting in gradual exposure of transplanted cell and their associated antigens to the host tissue. After a period of time, generally 3-8 weeks of exposure, the host can develop immune ignorance to the transplanted cells. Exposure times may be increased or decreased according to the degradation rate of the degradable HFM. Following development of immune tolerance to the transplanted cells and degradation of the HFM, functional cell transplantation can be carried out.

In other embodiments, delivery of other growth and development factors or other active agents such as angiogenesis promoting factors, for example, Vascular endothelial growth factor (VEGF), bradykinin, and other angiogenesis promoting factors, can be combined with any of the above-described embodiments to promote angiogenesis in the transplantation zone, before, during, and/or after functional cell transplantation, and improve the chances of survivability of the transplanted cells.

In still other embodiments, to further maximize the viability and functionality of transplanted cells, growth factor delivery may be applied in conjunction with any of the above-described embodiments. For example, in nervous system repair, candidate growth factors include GDNF, BDNF, NT3, bFGF, FGF4, EGF, neural survivor factor-1, nerve growth factor, TGF-b3, FGF8 isoform b, and other neurotrophic factors and the like as well as factors that suppress the inhibition of axonal growth present in the adult brain. Other agents that can be combined with the above-described embodiments include free-radical scavengers and anti-apoptotic agents.

The present disclosure may be better understood with reference to the following examples:

### EXAMPLE 1

### Transplantation of Functional Cells After Acute and Sub-Acute Inflammatory Period Can Increase the Survival Rate of Transplanted Cells

To examine the role of acute and sub-acute inflammatory responses (1-4 week after injury) on the survival of transplanted cells, a re-accessible transcranial device assembled from a non-degradable polyurethane access port and a non-degradable semi-permeable polyurethane HFM for the delivery/encapsulation of dopaminergic PC12 cells or bone marrow stem cells into the striatum of 6-OHDA lesioned adult rat striatum. For the group transplanted at the acute inflammatory stage, PC12 cells or bone marrow stem cells were transplanted at the time of the device implanted. For the group transplanted at the sub-acute inflammatory stage, cells were transplanted at 5th day after the implantation of the device. For the group at chronic inflammatory stage, cells were transplanted at 4 weeks after device implantation. After 8 weeks of cell transplantation, encapsulated cells are stained with live/dead staining kit to examine the survival rate of transplanted cells. The survival rate of transplanted PC12 cells was increased from 74±7.2% (transplanted at the time of device implantation) or 84±5.7% (transplanted at the 5th day after device implantation) to 96±4.3% (transplanted at 4 weeks after device implantation). Animals that transplanted at 4 weeks after device implantation shown better functional recovery when compared with other two groups. The survival rate of transplanted bone marrow stem cells was increased from 58±8.9% (transplanted at the time of device implantation) or 71±6.1% (transplanted at the 5th day after device implantation) to 83±4.9% (transplanted at 4 weeks after device implantation). This data clearly demonstrated that acute or sub-acute inflammatory responses could greatly impact the survival of transplanted cells. Therefore, a technique was developed to avoid the grafting-associated acute and subacute inflammation response using a transcranial HFM based device.

### EXAMPLE 2

### Scar-Free Space may Increase the Long-Term Survival and Migration of the Transplanted Cell in Brain Tissue

To examine the role of scar surrounding the transplantation cells on the long-term survival and migration of the transplanted cells, such as human stem cell derived dopaminergic neurons. A re-accessible transcranial device showing FIG. 1 assembled from a non-degradable polyurethane access port, a short segment of non-degradable polyurethane HFM and a segment of biodegradable semi-permeable chemically chitosan HFM for the creation of a scar-free space inside brain tissue for future transplantation of functional cells, human stem cell derived neurons. Postnatal day 1 immature astrocytes or their derived soluble factors were mixed with gels and attached onto a polystyrene coil and encapsulated into the degradable HFM lumen at the time of device implantation. Four weeks later, coil inserts were removed from the device. Two weeks later, when the degradable HFM is degrade, and a scar-free space is created for functional cell transplantation. The survival of the transplanted neurons, the behavioral and functional recovery of the adult male PD rats that undergo the two strategies of implantation with or without co-transplanted immature astrocytes (P1) will be compared and compared.

### EXAMPLE 3

### The Viability of Transplanted Stem Cells can be Improved After the Exposure of Hypoxia Simulating Agents to Stem Cells Before Transplantation

Human umbilical cord stem cells were exposed to 50 uM/ml ethyl 3,4-dihydroxybenzoate for 12 hours before transplanted into brain tissue to improve their tolerance to hypoxia condition at the injection site after transplantation procedure. Ethyl 3,4-dihydroxybenzoate can increase glucose uptake and glycolytic metabolism of cells. Increased glucose uptake and glycolytic metabolism is an important compensatory mechanism that allows ATP production to be maintained. Such an approach also increases resistance of the transplanted cells to metabolic poison. The resulting transplanted cells will be adjusted to a deficiency of oxygen and, thus, more resistant to transplantation procedures. The survival rate of the transplanted cells is higher for ethyl 3,4-dihydroxybenzoate treated group than that for untreated group.

### EXAMPLE 4

### A Glial Scar-Free Implant-CNS Tissue Interface can be Created by Blocking CSPG Biosynthesis

Following adult central nervous system (CNS) injury, resident cells and blood-derived inflammatory cells become activated and migrate to the lesion site, eventually form a dense glial scar that is believed to play a significant role in inhibiting CNS regeneration. The inhibitory nature of the glial scar is due not only to the existence of inflammatory cell types, but also to the ECM molecules that are derived from them. In particular, chrondroitin sulfate proteoglycan (CSPG), a family of ECM molecules secreted by astrocytes, has been shown to upregulate in the glial scar and inhibit neurite extension, and the digestion of CSPG has enhanced axonal regeneration after spinal cord injury. Blocking CSPG biosynthesis may be a potential means to ameliorate CNS glial scar formation. An implantable hollow fiber membrane (HFM) nerve guidance channel has been selected as a model system attributing to its demonstrated efficacy in guiding CNS regeneration. CSPG inhibitor 4-nitrophenyl-β-D-xylopyranoside (PNPX) was incorporated into the HFMs during the fabrication process. The inflammatory responses of adult brain tissue to the HFMs with or without PNPX were evaluated and compared.

Polyacrylonitrile/polyvinylchloride copolymer (PAN/PVC) was used as a model non-degradable polymer as a carrier for PNPX delivery, and poly(DL-Lactide-co-glycolide) (PLGA) 50/50 copolymers was employed as a model degradable polymer for PNPX delivery. Polymer-PNPX was dissolved in dimethyl sulfoxide (DMSO). 1% phthalized-chitosan was added into the solution to enhance the PNPX loading efficiency. PNPX loaded HFMs were fabricated using a wet phase-inversion technique. PNPX loading efficiency and releasing profiles were studied in vitro using HPLC. PNPX loaded HFMs were implanted stereotactically into adult Fischer 344 male rat brains (+0.2 mm Bregma, +3.0 mm lateral, 10 mm depth from dura, with nose bar at −3.3 mm). Animals were sacrificed for immunohistological analysis at 2, 4, and 8 weeks (n=7 each time point) following implantation. Horizontal sections of the fixed brains were obtained at a thickness of 50 um using a vibratome. Indirect immunohistochemistry were performed on the sections using antibodies against CSPG, GFAP, Neurofilament, OX-42, ED-1, and Reca-1 to study the glial response, neuronal reaction, foreign body reaction, and angiogenesis to the PNPX loaded HFMs. 4′,6′-diamidino-2-phenylindole hydrochloride (DAPI) was used for nuclei staining on all the sections.

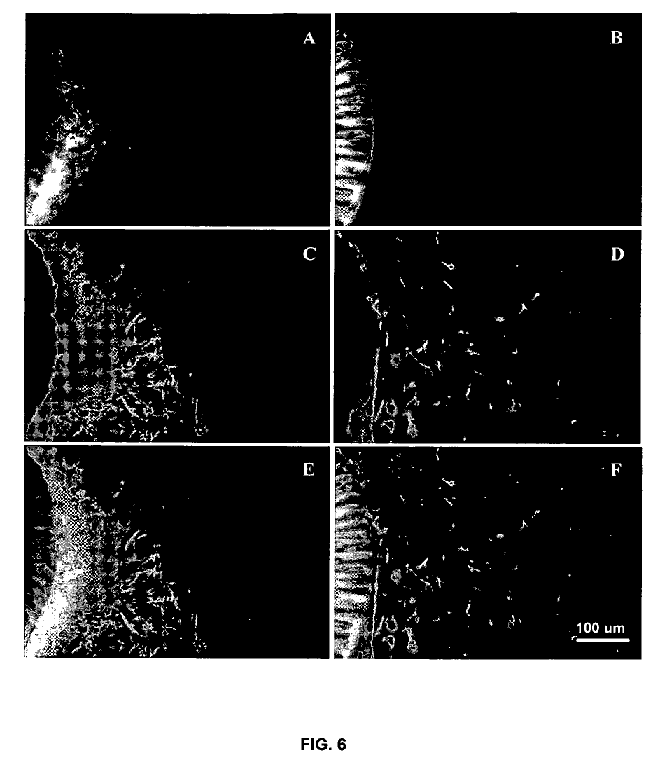

PNPX loaded HFMs showed a steady release of PNPX up to four weeks in vitro. The extent of astrocytic scarring was examined using anti-GFAP (for astrocytes) and anit-CSPG antibodies. Increase in GFAP levels as a result of astrocytes proliferation and hypertrophy may occur during glial scarring response. As shown in FIG. 6, compared to the blank control HFM, constant release of PNPX into local brain tissue significantly (P<0.05) suppressed the glial scarring response in the vicinity of the PNPX loaded HFM. No significant difference was found in neuronal reaction, foreign body reaction, and angiogenesis between the PNPX loaded HFMs and the control HFMs. Studies in progress are evaluating axonal outgrowth and regeneration in PNPX loaded HFM guidance channel using a spinal cord injury model.

Adult brain tissue reaction to PNPX loaded HFMs (A, C, E) or non-loaded HFMs (B, D, F). (A), (B): CSPG staining pattern; (C), (D): GFAP staining pattern for astrocyte; (E), (F): overlapped pattern of CSPG and GFAP. Blue is DAPI staining for nuclei. The number of reactive astrocytes and the amount of CSPG surrounding the PNPX delivery zone is significantly (P<0.05) lower than that for the control, suggesting that PNPX may suppress glial scarring response after CNS injury.

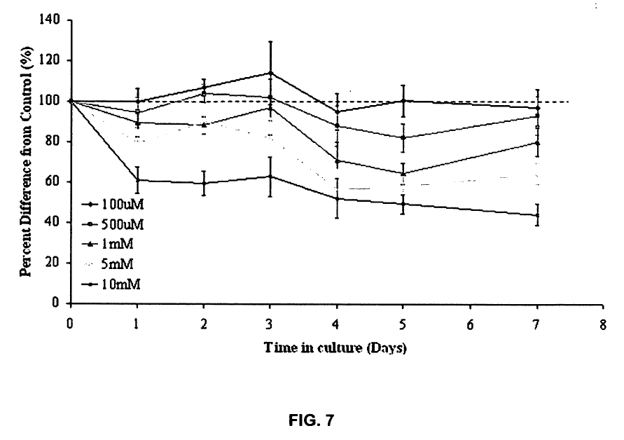

FIG. 7 depicts toxicity of PNPX to human mesenchymal stem cells.

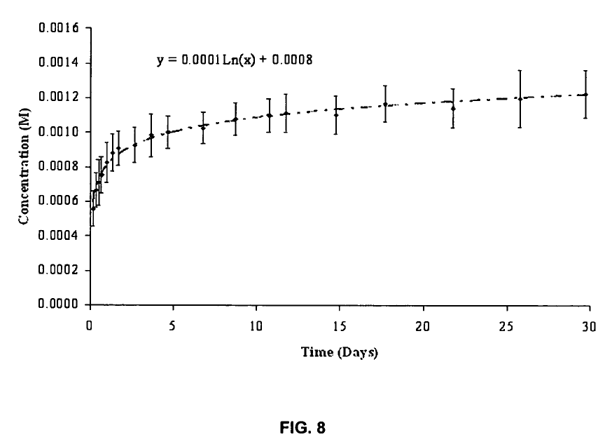

FIG. 8 depicts accumulated PNPX release from PNPX loaded HFM.

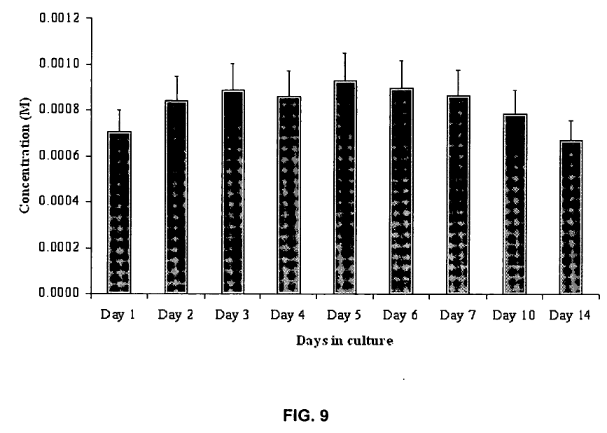

FIG. 9 depicts 24 hour release from PNPX loaded HFMs.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the disclosure so further described in such appended claims.

What is claimed is:

1. A method for improved viability of cells comprising:
providing a device comprising a body, an access port, and an insert, said body comprising a selectively permeable hollow fiber membrane, said insert comprising one or more scar suppression agents;
implanting said device whereby said scar suppression agent is delivered.

2. The method of claim 1, wherein said scar suppression agent comprises 4-nitrophenyl-β-D-xylopyranoside.

3. The method of claim 1, wherein said selectively permeable hollow fiber membrane is degradable.

4. The method of claim 1, wherein said insert further comprises one or more transplanted cells.

5. The method of claim 4, wherein said transplanted cells include stem cells.

6. The method of claim 4, further comprising developing immune tolerance in said transplanted cells.

7. A method for improved viability of cells comprising:
providing a device comprising a body, an access port, and an insert, said body comprising a selectively permeable hollow fiber membrane, said insert comprising one or more scar suppression agents and one or more transplanted cells;
implanting said device whereby said scar suppression agent and said transplanted cells are delivered.

8. The method of claim 7, further comprising increasing hypoxia tolerance of said transplanted cells.

9. The method of claim 7, wherein said selectively permeable hollow fiber membrane is degradable.

10. The method of claim 7, wherein said transplanted cells include stem cells.

11. The method of claim 7, further comprising adding additional transplanted cells.

12. A device for delivery of scar suppression agents comprising:
a body, said body including a proximal end and a distal end, said body defining a cavity;
an access port, said access port located at said proximal end of said body; and
an insert, said insert having a proximal end and a distal end, said insert configured to be inserted into said cavity of said body, said insert comprising one or more scar-suppression agents.

13. The device as defined in claim 12, wherein said insert further comprises one or more functional cells.

14. The device as defined in claim 13, wherein said functional cells have hypoxia tolerance.

15. The device as defined in claim 13, wherein said functional cells comprise stem cells.

16. The device as defined in claim 12, wherein said scar suppression agent comprises 4-nitrophenyl-β-D-xylopyranoside.

17. The device as defined in claim 12, wherein said selectively permeable hollow fiber membrane is degradable.

18. A device for delivery of scar suppression agents comprising:
a body, said body including a proximal end and a distal end, said body defining a cavity;
an access port, said access port located at said proximal end of said body; and
an insert, said insert having a proximal end and a distal end, said insert configured to be inserted into said cavity of said body, said insert comprising one or more scar-suppression agents and one or more transplanted cells.

19. The device as defined in claim 18, wherein said transplanted cells comprise stem cells.

20. The device as defined in claim 18, wherein said transplanted cells have hypoxia tolerance.
